(19) United States
(12) Patent Application Publication
(10) Pub. No.: US 2011/0159102 A1
(43) Pub. Date: Jun. 30, 2011

(54) Apparatus and Method for Continuous Production of Spherical Powder Agglomerates

(75) Inventors: Thorsten Hartmann, Berlin (DE); Bernd W. Müller, Flintbek (DE); Hartwig Steckel, Kiel (DE)

(73) Assignee: Pharmatech GmbH, Flintbek (DE)

(21) Appl. No.: 12/738,469

(22) PCT Filed: Oct. 13, 2008
(86) PCT No.: PCT/EP2008/008655

§ 371 Date: Sep. 21, 2010

(30) Foreign Application Priority Data
Oct. 18, 2007 (DE) 10 2007 049 931.2

(51) Int. Cl. A61K 9/14; B24B 31/00; B02C 17/02

(52) U.S. Cl. 424/489; 241/24.15

(57) ABSTRACT

The disclosure relates to a process for continuously producing spherical powder agglomerates, in which morphologically irregular starting agglomerates of micronized pulverulent particles are rounded off continuously by application to a surface induced to vibrate.

### BACKGROUND OF THE INVENTION

Micronized substances, i.e., powdered products with the distribution of particles of the size in the range 1 to 10 μm find nowadays a variety of applications. However, in all cases, there are substantial technical issues related to the degree of their easiness to be handled and exact control of dosing due to the high tendency of individual particles to further agglomerate, because their automatic adhesion forces, here mainly the Van-der-Waals forces, considerably exceed the force of gravity exerted on them.

Micronization of powders occupies an important place in the field of inhalative application of active pharmaceutical ingredients, because in order to ensure sufficient pulmonary availability, the relevant particles must have a diameter of approximately 1 to 5 μm. Especially in this area, a precise dosage of often very small volumes plays an important role. A common method to achieve the requisite sufficient flowability of preparation is the production of so-called interactive powder mixtures, i.e., the conscious binding of micronized active particles to an inert carrier. In most cases, this is performed using lactose particles in a particle size range from 50 to 200 μm, whereby the obtained active substance mixture in comparison to the pure micronized active substance possesses a significantly improved fluidity. Using suitable powder inhalers, by targeted air turbulence within the device, the active substance is again separated from the carrier and can thus be inhaled when the carrier remains in the device itself and/or in the mouth or pharyngeal cavity of the user. With this design however, the tendency to bond between the active substance and the carrier is so high that a large part of the active particle remains bound to the carrier, and therefore (depending on the particular type) remains unused in the powder inhaler or is swallowed by the user rather than to reach the lungs as is desired.

In the search for alternatives to interactive powder mixture, it turned out that the selective agglomeration of micronized active ingredients can provide an alternative to interactive powder mixture with carrier particles. This process includes the production of spherical agglomerates (pellets) of pure active substance particle or with the addition of some micronized excipient that is stable enough to allow the technical handling, but unstable enough to allow in turn a pulmonary delivery by re-dispersion in a suitable powder inhaler according.

### STATE OF THE ART

The patent SE19930003214 19,931,001 describes the preparation of such an agglomerate referred to as “soft pellets”, where starting pellets are produced by forcing micronized powder through a sieve. The powder is here supplied with a screw conveyor and the generated starting pellets are rounded, in batches, in a rotating pan and then sorted through a sieve to obtain the percentage of pellets of a predetermined size range.

In contrast, the device of the invention and the invented method are based on a completely different type of rounding. Instead of the batch-wise production in small quantities (up to 500 g) in a rotating vessel, the invented device and method use for this purpose a vibrating surface.

Advantages can be seen here:
In the possibility to forego the charge-by-charge production and, instead, to continuously produce;
under the given conditions, in an increase in the yield by a more targeted rounding of individual starting pellets;
in the time saved due to a shorter rounding time; in the following examples, the difference is represented by the time of usually between 30 and 90 seconds as compared to 120 to 1200 seconds indicated in SE19930003214 19931001.

In addition, in terms of their hardness or breaking strength, the pellets as described in SE19930003214 19931001 are within a range from 0.5 to 100 nm. In contrast, the invented device and the method of the invention allow the production of spherical pellets with a hardness and fracture resistance of less than 0.5 nm.

The method as described in SE19930003214 19931001 can also be found, in an exact or similar form, in the following patents: GB20000012261 20000519; GB 19940004945 19940315; SE19970000133 19970120; SE19970000134 19970120; SE19970000135 19970120; SE19970000136 19970120 so that all of them substantially differ from that described by our invention.

The patent CH19900000266 19900129 describes a device and a method for dosing small amounts of poorly flowing powders.

In contrast to this, the invented device and the method of the invention focus not on the filling or the dosing, but rather on the production of targeted rounded powder agglomerates or pellets. Moreover, in the present invention, the goal is especially the clear spherical morphology of the resulting powder agglomerates.

In addition, the CH19900000266 19900129 concerns merely a formoterol-lactose mixture and does not disclose the universal nature of the invention and the inventive device.

A number of other patents also deal with the filling and transportation of (partially agglomerated) substances. They all significantly differ from the present invention, because essentially the transportation dosing requires a completely different kind of construction of the device and (if they apply vibration or oscillation), they have different frequency ranges.

Specifically, these are:

The GB19950015340 19950726 describes the dispensing of powders, where the volume to be filled is achieved using a vibrating feed hopper working in the frequency range of 1 to 1000 Hz. In contrast to the invented method and the device of the invention, apart from the similar frequency range there exist no matches.

The US20030434009 2003050 describes an apparatus for dispensing (dosing) powders, in which the filling process is allowed or supported by a vibration of the hopper in the frequency range of 10 to 1,000,000 Hz. It is mentioned explicitly that, in this case, a powder flow without any aggregation, i.e., agglomeration is to be generated.

The US19970100437P 19970721 relates to the transport of powders, where powder-transporting parts oscillate vertically in the frequency range from 1000 to 180,000 Hz and horizontally, in the frequency range from 50 to 50,000 Hz.

The US19960638515 1996042 is limited to the transport and a subsequent dosing of powders. The device has one or two screens. These screens oscillate at a frequency in the range of 1 to 500 Hz. No value whatsoever is placed on the morphology of the thus produced agglomerates. Furthermore, it is only assumed that only an undefined mixture of small agglomerates and individual particles is generated.

The US19850713697 19850319 describes a device that should allow the transport and subsequent dosing of powders, wherein the powder flow should be ensured by means of a vibrating transportation surface. There is no attention given to the emerging powder agglomerates and their morphology.

The SE20040000282 20040209 relates to the measurement and filling of powders. Oscillation in the form of ultrasonic vibrations or similar vibration is used here only in support of the filling process.

The technical task that underlies the present invention is to provide a new method and a new device for a continuous production of spherical powder agglomerates or pellets, which overcome the disadvantages of the prior art.

This technical task is resolved by a method according to claim 1 and a device according to claim 10.

Preferred embodiments are the subject of the dependant claims.

The present invention is represented by an apparatus and a method for a continuous production of spherical powder agglomerates (pellets). The expected properties of the resulting spherical agglomerates (pellets) should include a nearly perfect roundness, so the best possible flow, consequently, a good ability to be dispensed in accurate dosage, sufficient stability for the technical handling and, finally, a satisfactory ability to be re-dispersed in pulmonary application.

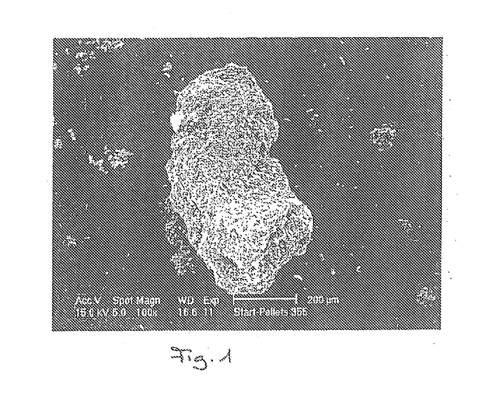

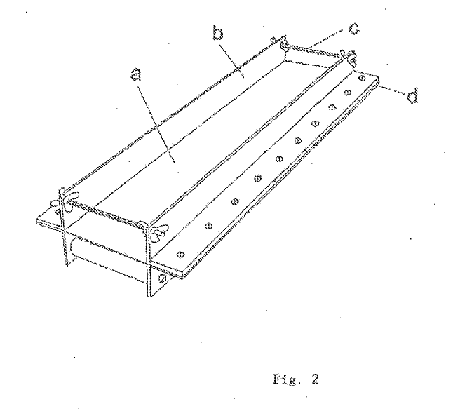

For this purpose, the powder to be treated, whose individual particles are preferably micronized, i.e., they are present with an average diameter of 5 μm, is formed by forcing it through a sieve or sieve-similar device to become morphologically heterogeneous starting pellets [FIG. 1]. These can be either pure powders, preferably pharmaceutical active substances for pulmonary application, as well as mixtures of different substances in powder form, preferably mixtures of pharmaceutical active substances for the pulmonary administration that act as a substance that is inert as regards a direct pharmacological effect, preferably inert as far as possible, that is essentially inert, i.e., a filler such as lactose in different circumstances. The size of individual particles, of which the starting pellets are formed, can vary over a range of 1 to 50 μm. Depending on the mesh size of the used sieve or sieve-similar device, the size of the starting pellets varies between 20 and 1000 μm. Therefore, the choice of the mesh size and thus the determination of the size of both the starting and ultimately the final pellets should be made dependent on the substances used. In more concrete terms, this means that substances with a very high auto-adhesion propensity for a final pellet size of, for example, 50 μm, can still have an insufficient flow capacity and, in this case, in spite of an ideal rounding, a bigger diameter of the final pellet should be endeavored. On the other hand, if we choose too large a mesh or mesh-similar device, mixtures with a higher proportion of larger particles, for example, due to insufficient adhesion, they can form only very unstable starting pellets, which can consequently not be made round at all but, rather, disintegrate already in the experiment.

A design illustrated in FIGS. 2 to 4 demonstrates a method to round the starting pellets to form final pellets. The core of the device is represented by a metal foil [a], which in the present case is a cold-rolled stainless spring steel (1.4310 DIN X12 CrNi17 7) of the dimensions 15 cm×50 cm×0.1 mm, which is held between two aluminum angles [b] with the help of 10 screws. Two metal rods with wing nuts [c] offer the possibility of regulating the tension of the metal foil. In the area of the places of contact to the aluminum angles, there is located—above and below the metal foil—a silicone layer [d] in the present structure 1 mm thick, ensuring a secure fixing and additional cushioning.

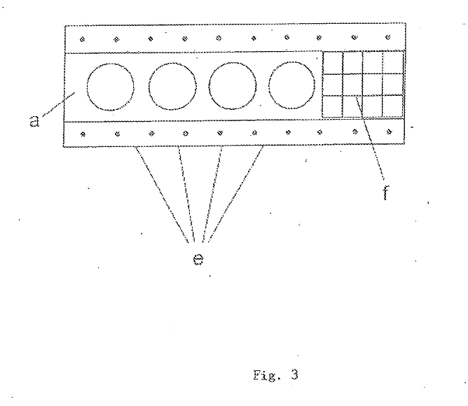

At the bottom of the device [FIG. 3], frequency generators [e] are directly attached to the metal foil at regular intervals. In this particular design, these are four soundpad NXT® foil speakers, which are fixed to the foil with a circular-shape, double-surface adhesive tape, where, however, any other type of oscillation-generating components is possible. A marked field [f] is recessed in one of the two ends of this device.

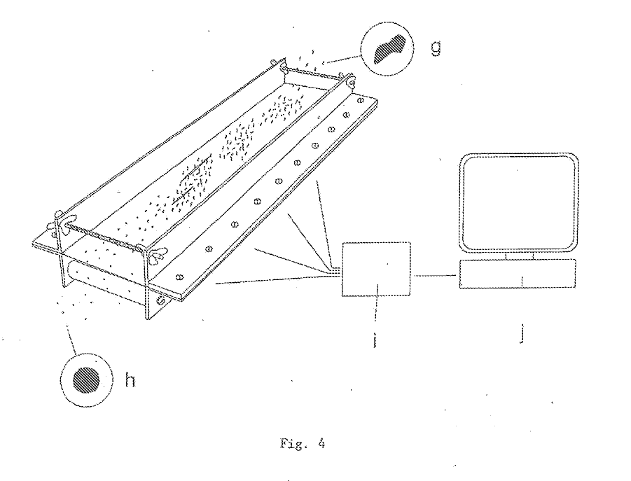

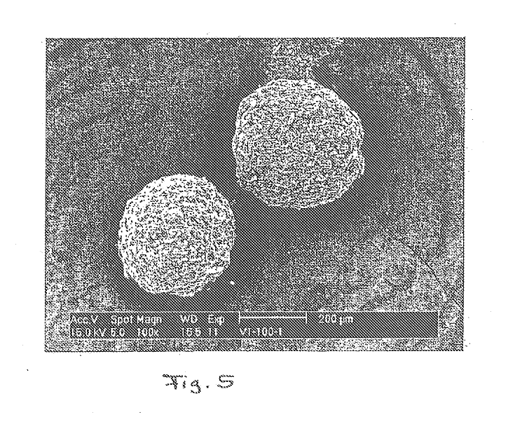

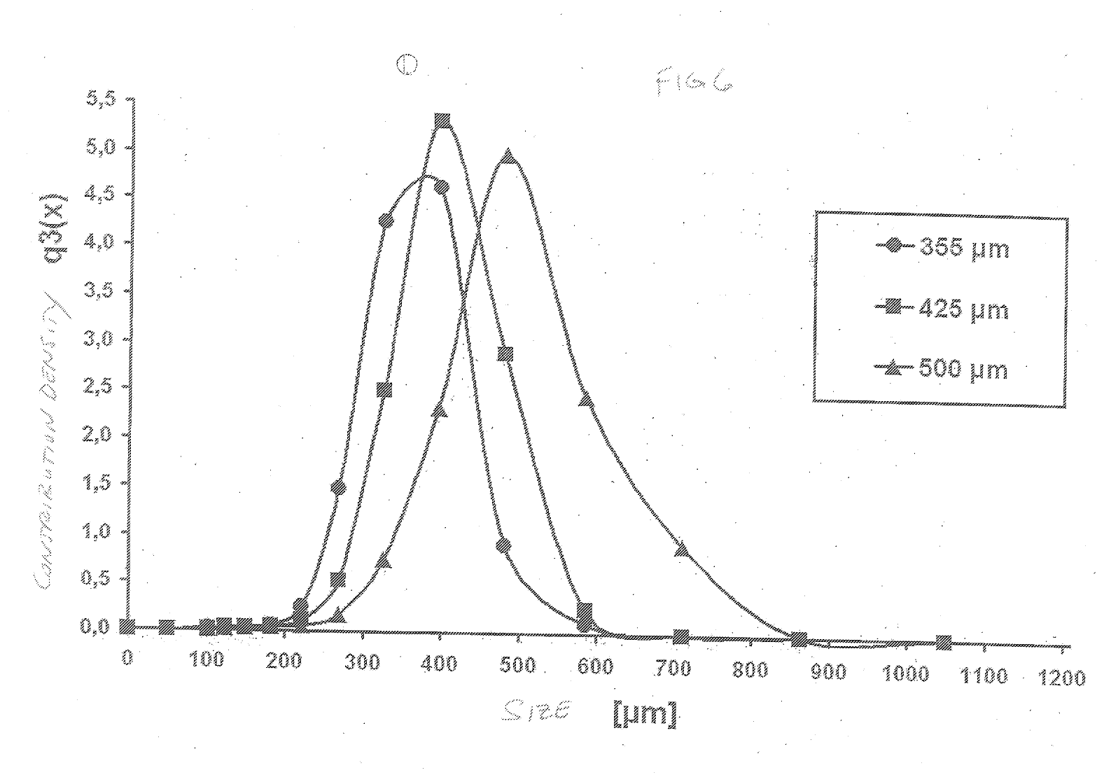

Using a combination of a control unit [i] and a computer [j], the fixed-frequency generators [FIG. 4] are brought into the desired oscillation, which has preferably a frequency of 50 to 150 Hz and, due to its firm fixation, is transferred directly to the metal foil. The entire device is placed on a solid surface with a fixed slope—depending on the length of the vibrating surface, the applied vibration frequency and the resulting deflection; theoretically, angles from 0° to 89° are conceivable, but preferably between 2° and 10°. If we now place the preformed starting pellets [g] in the proximal region [f], due to the vibrating surface the pellets assume a rounded shape and, at the same time, to move to the distal end of the device. Here, therefore, the spherical final pellets [h] are collected and are available for further processing steps such as a possible sorting or direct filling. In this way, a continuous production of spherical powder pellets is made possible [FIG. 5].

### EXAMPLES

Micronized disodium cromoglycate (DSCG) is agglomerated by forcing it through a sieve with a mesh size of 355 μm. Scanning electron micrographs of these starting pellets are shown in [FIG. 1] (See further above). These starting pellets are fed to the previously described apparatus in such a fashion that the substance is sieved directly to the metal foil. As described, the metal film has a total length of 50 cm and a width of 15 cm. The foil oscillates with a computer-controlled frequency of 100 Hz. The slope of the overall setup to the surface is approximately 7°. The pellets received at the distal end of the device have a spherical morphology, which is shown in [FIG. 5]. Subsequent sizing using a sieve with a mesh size of 200 μm shows only a marginal loss in the form of fine material so that, in relation to the starting quantity, we obtain a yield in the range of 90% to almost 100%. If we study the pellet morphology in a more detailed manner using image analysis (L2001, Leco GmbH, Kirchheim, Germany), as illustrated by the scanning electron micrographs [FIG. 5], the pellets emerge complete rounded. Relevant parameters represent this aspect ratio (length ratio, ideally=1), roundness (RN=(4n*area)/(circumference)², ideally=100, or real=95 due to the pixel matrix of the measurement system), roughness (RG=ratio of volume/convex volume; ideally=1), and width (shortest Feret's diameter). In the described example, the resulting pellets have a length ratio of 1.16, with a roundness 87.7 and a surface roughness of 1.019. The average width of the agglomerates is at approximately 366 μm. Using impactor tests, aerodynamic characterization of the obtained pellets shows a very satisfactory redispersibility into individual particles, which suggests a good availability of the active substance DSCG for pulmonary delivery.

2. Micronized disodium cromoglycate (DSCG) is treated as described in Example 1, with the difference that the resulting spherical pellets are placed on the metal foil, which oscillates a frequency of 100 Hz, two more times; thus they travel the threefold distance on the foil. As expected, this results in an even more rounded shape, with a length ratio of the resulting pellets of 1.15, with a roundness of 88.6 or roughness of 1.016. A detailed aerodynamic characterization also reveals an influence of the changes in experimental conditions such that the share available for pulmonary delivery slightly decreases, but continues to remain within a very satisfactory range.

3. Micronized disodium cromoglycate (DSCG) is treated as described in Example 1, with the difference that the metal foil of the device oscillates at a frequency of 50 Hz. The result is in a comparable range, whereas the quality, as for roundness and the roughness slightly decreases, however, for the benefit of improved redispersibility, and therefore improved pulmonary availability in the aerodynamic characterization.

4. Micronized disodium cromoglycate (DSCG) is treated as described in Example 1, with the difference that the metal foil does not constantly oscillate with a frequency of 100

Hz throughout the manufacturing process, but the frequency is modulated. More specifically, this means that the frequency goes through a range of 100 to 20 Hz over a period of one second in increments of 1 Hz. After a second, the frequency jumps immediately back from 20 Hz to 100 Hz, and in the period of the next second it again reaches 20 Hz in steps of 1 Hz. This variant of the method provides also results comparable with the other examples.

5. Micronized budesonide is treated similarly to that described in Example 1 with comparable results.

6. Micronized fluticasone propionate is treated in a fashion similar to that described in Example 1 with comparable results.

7. Micronized salbutamol sulphate is treated similarly as described in Example 1 with comparable results.

8. A mixture of micronized formoterol fumarate and lactose in the ratio 1:50 is treated similarly to that described in Example 1 with comparable results.

9. A mixture of micronized budesonide, formoterol fumarate and micronized lactose in the ratio 2:1:50 is treated similarly to that described in Example 1 with comparable results.

10. A mixture of micronized fluticasone propionate, micronized salmeterol xinafoate and lactose in the ratio 2:1:50 is treated in a fashion similar to that described in Example 1 with comparable results.

11. Pharmacologically largely inert substances may also be treated as described in Example 1. Here, the point is logically not to achieve a good pulmonary availability, but rather to improve the manageability and controllability of these substances. Consequently, lactose treated in a fashion similar to that in Example 1 also provides spherical pellets that meet the above criteria.

12. Analogously to Example 10, one obtains comparable results in the treatment of mannitol.

The figures show:

FIG. 1: An example of morphologically heterogeneous starting pellets

FIG. 2: Schematic design of the device, top

FIG. 3: Schematic design of the device, bottom

FIG. 4: Schematic design of the device, overview

FIG. 5: An example of two resulting spherical pellets

1. A method for a continuous production of spherical powder pellets, comprising:
producing morphologically irregular starting pellets made from micronized powder particles by forcing powder to be treated with particles having a size ranging from about 1 to about 50 μm through a mesh having a mesh size ranging from about 20 to about 1000 μm to provide morphologically irregular starting pellets having a size ranging from about 20 to about 1000 μm; and
continuously feeding the morphologically irregular starting pellets on an inclined oscillating surface to provide individual final pellets having a spherical shape and a size ranging from about 20 to about 1000 μm.

2. The method according to claim 1, wherein the powders to be treated are pure mono substances or a mixture of more substances in ratios ranging from about 1:1 to about 1:1000.

3. The method according to claim 2, wherein the powders to be treated are pharmaceutically active substances, both as a mono substance and in form of a mixture with pharmacologically inert or essentially inert substances.

4. The method according to claim 2, wherein the powders to be treated are a mixture of several different pharmaceutically active substances.

5. The method according to claim 2, wherein the powders to be treated are a mixture of one or more different pharmaceutically active substances and pharmacologically inert or essentially inert substances.

6. The method according to claim 1, wherein the powders to be treated are pharmaceutically active substances designed for inhalation.

7. The method according to claim 1, wherein the mesh size ranges from about 100 to about 1000 μm.

8. The method according to claim 1, wherein the individual final pellets have a size ranging from about 100 to about 1000 μm.

9. A device for carrying out the method according to claim 1, wherein the inclined surface, that can be made to oscillate, is made from a material selected from the group consisting of metal, plastic or ceramic, and has a thickness ranging from about 0.001 to about 10 mm, as well as a length and a width each ranging from about 10 to about 200 cm.

10. The device according to claim 9, wherein the generation of the oscillation occurs on an electromagnetic basis by means of a frequency generator.

11. The device according to claim 9, wherein the generation of the oscillation occurs mechanically by means of a frequency generator.

12. The device according to claim 9, wherein the generated oscillation lies within a frequency ranging from about 10 to about 1000 Hz.

13. The device according to claim 10, wherein all the frequency generators used generate the same frequency.

14. The device according to claim 13, wherein the frequency remains constant throughout the production.

15. The device according to claim 13, wherein the frequency can be modulated so that the frequency runs through varying frequency ranges.

16. The device according to claim 9, wherein the mesh size ranges from about 100 to 500 μm.

17. Spherical powder pellets obtained by a method according to claim 1.

18. The device according to claim 12, wherein the generated oscillation lies within a frequency ranging from about 50 to about 200 Hz.

19. The device according to claim 9, wherein the thickness of the inclined surface ranges from about 0.025 to about 0.5 mm.

20. The method according to claim 1, wherein the individual final pellets have a size ranging from about 100 to about 500 μm.
